(19) United States
(12) Patent Application Publication
(10) Pub. No.: US 2009/0042284 A1
(43) Pub. Date: Feb. 12, 2009

(54) Method for labeling/separation of cells and agent for labeling/separation of cells

(76) Inventors: Shunro Tachibana, Fukuoka (JP); Katsuro Tachibana, Fukuoka (JP)

Correspondence Address: JORDAN AND HAMBURG LLP, 122 EAST 42ND STREET, SUITE 4000, NEW YORK, NY, 10168, US

(21) Appl. No.: 11/988,189

(22) PCT Filed: Jul. 4, 2005
(86) PCT No.: PCT/JP2005/012358

§ 371 Date: Feb. 13, 2008

(51) Int. Cl. C12N 5/06; C07K 16/00

(52) U.S. Cl. 435/325; 530/387.1

(57) ABSTRACT

Disclosed are a cell labeling/separation method and a cell labeling/separation agent capable of identifying a target cell in a simplified manner while achieving a significantly high degree of accuracy in cell separation. The method comprises adding a micro- or nano-capsule 1 encapsulating gas 2, into a solution containing a target cell 3 therein, and stirring the obtained mixture to allow the target cell 3 to be bound to an outer surface of a wall of the micro- or nano-capsule through an antibody 4, whereby only the target cell 3 is floated up and separated.

### TECHNICAL FIELD

The present invention relates to a cell labeling/separation method adapted to allow a target cell to be floated up and separated from other cells, and a micro or nano bubble capsule-based cell labeling/separation agent for use in the method.

### BACKGROUND ART

Heretofore, as a method of separating a specific cell, such as a leukemic cell or an immune cell, from other cells, there has been known a centrifugation technique utilizing a difference in specific gravity between a white blood cell and a red blood cell. In case of separating various types of cell, there has also been know a technique of mixing a solution having a given specific weight density with a sample, and separatingly precipitating a specific cell in an upper or lower solution layer generated in a test tube through centrifugation. This technique is a simple and accurate way to separate a specific cell from plural types of cells different in weight or specific density.

However, the above technique cannot be used for separating two types of cells similar in size or specific weight density, from each other. There have also been known various cell labeling/separation techniques of recognizing each of different types of cells (e.g., a subset of lymphocytes) using a fluorescent antibody and an optical laser device, and separating the cells individually by means of laser light or magnetic energy, such as a cell sorter, a microarray scanner, a FACSCan (Becton Dickinson Immunocytometry Systems), a laser trapping technique and a flow cytometry technique. Other cell labeling/separation technique includes: a technique of identifying a cell in combination with a labeling antibody, such as FITC, PE, PerCP, PerCP-Cy5.5, APC, PE-Cy7 or APC-Cy7; a bead assay using a functional polystyrene particle (Moritex Co.), a monoclonal antibody (CD4, CD8, etc.) or the like; a technique using a magnetic bead reagent; an enzyme-linked immunosorbent assay (ELISA); and an electrophoresis (see the following Patent Publications 1 and 2).

[Patent Publication 1] JP 2002-181781A

[Patent Publication 2] JP 09-61436A

[Patent Publication 3] U.S. Pat. No. 6,676,963

[Patent Publication 4] U.S. Pat. No. 6,528,039

### DISCLOSURE OF THE INVENTION

### Problem to be Solved by the Invention

The conventional cell labeling/separation techniques involve large-scale/costly equipment and complicated sample processing operations. Moreover, a test operation and a separation process are cumbersome and complicated, and it is necessary to take time for identification and separation.

As to assay for diseases prevalent in developing countries, such as AIDS, SARS, cholera and malaria, it is difficult to use the above conventional techniques, in view of cost, testing expertise and equipment. The conventional techniques are also required to collect a relatively large amount of sample (e.g., blood sample) at once so as to ensure accuracy in cell labeling and separation, which will impose a strain on a patient. In the field of research on cancers, antibodies and genes, a cell labeling/separation technique requiring only a small amount of sample and having desired simplicity and accuracy is not viable unless costly equipment is used. Further, in the fields of regenerative medicine (embryo-stem (ES) cell, stem cell, etc.) and cloning, there is the need for identifying and separating a cell while minimizing damages thereof.

In view of the above circumstances, the object of the present invention is to provide a cell labeling/separation method and a cell labeling/separation agent capable of identifying a target cell in a simplified manner while achieving a significantly high degree of accuracy in cell separation.

### Means for Solving the Problem

In the present invention, the above object is achieved by adding a micro- or nano-capsule encapsulating gas into a solution containing a target cell therein, and stirring the obtained mixture to allow the target cell to be bound to an outer surface of a wall of the micro- or nano-capsule through an antibody, whereby only the target cell is floated up and separated.

### EFFECT OF THE INVENTION

According to the present invention, irrespective of a size and specific weight density of each cell in a test sample, only a target cell can be floated up to an upper portion of the solution, and collected. A fluorescent material may be included in the micro- or nano-capsule, e.g., in such a manner that it is attached onto an outer or inner surface of the wall of the micro- or nano-capsule, or contained within the wall, so as to allow the target cell to be readily identified. The target cell will reach an uppermost portion of the solution based on a buoyancy of the micro- or nano-capsule encapsulating gas. Thus, the present invention makes it possible to separate a single cell from one million cells with a significantly high degree of accuracy in cell separation, as compared with the conventional separation techniques.

### BEST MODE FOR CARRYING OUT THE INVENTION

The present invention employs a micro- or nano-capsule 1 encapsulating gas 2, as a means to allow a target cell 3, i.e., a specific cell to be selectively separated from other cells, to be bound by an antibody 4, as shown in FIG. 1(a), and separated from other cells based on a buoyancy of the micro- or nano-capsule 1, as shown in FIG. 1(b).

The gas to be used in the present invention consists of gas non soluble in a liquid, such as propane gas, nitrogen gas or neon gas. The wall of the micro- or nano-capsule may be made of a lipid material, an albumin material or a polymer material. The antibody 4, such as a polyethylene glycol ligand or a monoclonal antibody, may be pre-bound onto the wall of the micro- or nano-capsule, so as to be bonded to a specific target cell.

The micro- or nano-capsule (e.g., having a diameter of 10 nm to 100 μm) encapsulating the gas is prepared in the same manner as that for a liposome, and an antibody to a target cell is bound to an outer surface of the wall of the micro- or nano-capsule (e.g., the number of antibodies is 10 to one billion/ml). The prepared micro- or nano-capsule is added into a solution containing the target cell therein, and the obtained mixture is stirred. Then, the mixture is left at rest for one minute to 24 hours. During the rest, the target cell is floated up. Thus, an upper portion of the solution can be collected to separate only the target cell from other cells. With a view to reducing the rest time, the mixture may be centrifuged at a low speed (e.g., 1000 rpm or less). The above operations may be repeatedly performed to obtain a higher degree of accuracy in cell separation.

As shown in FIG. 1(a), a fluorescent material 6 may be included in the micro- or nano-capsule 1. In this case, the fluorescent material 6 of the micro- or nano-capsule after being attached to the target cell will generate fluorescence so as to allow the target cell to be labeled therewith. While a conventional labeling technique of binding a fluorescent dye directly onto a surface of a target cell is likely to cause a cytotoxic effect, the present invention is free of such a risk.

The micro- or nano-capsule can be unbound from the surface of the target cell by applying thereto an ultrasonic wave (e.g., a frequency of 20 kHz to 10 MHz and a sound pressure of 10 W/cm² or less).

A micro- or nano-bubble developed as an ultrasonic contrast medium, or a gas-filled microsphere under research and development (see the aforementioned Patent Publications 3 and 4) may be used as the gas or the capsule in the present invention. The cell labeling/separation method of the present invention makes it possible to readily obtain information about a state of a patient's immune function, a type of bacterial infection, a degree of severity of a cancer, etc. Various types of monoclonal antibodies may be used as an identification marker to achieve the above functions. The present invention can also be used for separation of an ES cell or a stem cell.

### EXAMPLE 1

A blood sample was taken from a malaria patient. Then, a micro- or nano-capsule having an antibody to a malaria parasite was added into the blood sample, and the obtained mixture was stirred. Subsequently, a red blood cell bonded to the micro- or nano-capsule and separatingly floated up to an upper portion of the mixture was collected, and diagnostic information about disease state of the patient could be obtained from the number of abnormal red blood cells and microscopic pathological findings to determine a treatment policy.

### EXAMPLE 2

A blood sample was taken from an AIDS patient. Further, two types of micro- or nano-capsules each having an antibody to either one of CD4 and CD8 cell receptors (indicated by the reference numeral 5 in FIG. 1(a)), and a different fluorescently-labeling material was provided in each of the micro- or nano-capsules. Then, the prepared micro- or nano-capsules were added to the blood sample, and the obtained mixture was stirred. After an elapse of a given rest time, an upper portion of the mixture was collected, and subjected to fluorescence microscope observation, so that a progress of the disease, i.e., whether AIDS is in an active phase, could be determined.

### EXAMPLE 3

In the field of research on proliferation of leukemic cells and cancer cells, a specific type of cell can be labeled and separated according to the present invention to perform a cancer gene-expression analysis. For this purpose, several cell surface markers for a cultured cancer cell line were bound to an outer surface of a micro- or nano-capsule, so that several types of divided cells could be identified to clarify a cancer cell expression based on a combination thereof. In this case, the present invention makes it possible to effectively limit the number of cells to be collected, to a small value.

### BRIEF DESCRIPTION OF DRAWINGS

FIGS. 1(a) and 1(b) are explanatory diagrams of a cell labeling/separation method according to one embodiment of the present invention.

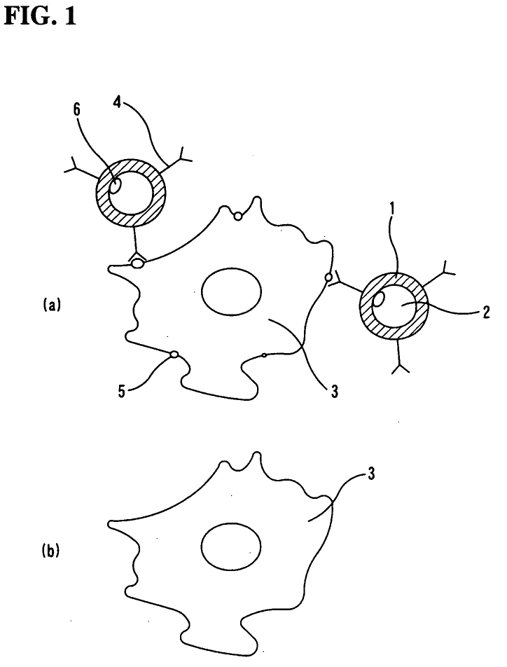

### EXPLANATION OF CODES

1: micro- or nano-capsule
2: gas bubble
3: cell
4: antibody
5: receptor
6: fluorescent dye

What is claimed is:

1. A method of labeling and separating a cell, comprising:
adding a micro- or nano-capsule encapsulating gas, into a solution containing a target cell therein; and
stirring the obtained mixture to allow said target cell to be bound to an outer surface of a wall of said micro- or nano-capsule through an antibody or receptor, whereby only said target cell is floated up and separated.

2. An agent for labeling and separating a cell, comprising a micro- or nano-capsule encapsulating a micro- or nano-bubble therein.

3. The agent as defined in claim 2, wherein said micro- or nano-capsule includes a fluorescent material.
